(19) United States
(12) Patent Application Publication
(10) Pub. No.: US 2010/0082070 A1
(43) Pub. Date: Apr. 1, 2010

(54) Osteosynthesis device

(75) Inventor: Ingolf Diez, Tuttlingen (DE)

Correspondence Address: MESMER & DELEAULT, PLLC, 41 BROOK STREET, MANCHESTER, NH, 03104, US

(73) Assignee: S.I.M.E.O.N. MEDICAL GMBH & CO. KG, Tuttlingen (DE)

(21) Appl. No.: 12/567,234

(22) Filed: Sep. 25, 2009

(30) Foreign Application Priority Data
Sep. 30, 2008 (EP) 08 017 211.7

(51) Int. Cl. A61B 17/80

(52) U.S. Cl. 606/286

(57) ABSTRACT

An osteosynthesis device comprises a plate 10 with at least one through hole 14 through which a screw may be screwed into a bone. The plate 10 comprises an insert 12 around the through hole 14, which consists of a material which enables the screw to self-cut a thread. A positive locking 20, 22 secures the insert 12 in the plate.

### BACKGROUND OF THE INVENTION

1. Field of the Invention

The invention relates to an osteosynthesis device which comprises a plate with at least one through hole through which a screw may be screwed into a bone, for bridging, in particular, bone fractures and for joining the bone with the plate, wherein the plate comprises an insert around the through hole, which consists of a material which is softer than that of the plate, into which the screw may be screwed while at least partially self-cutting a thread.

2. Description of the Prior Art

Such osteosynthesis plates are known, e. g. from DE 196 29 011 C2. WO 2008/077482 A1, too, describes an osteosynthesis device of the type discussed herein. DE 195 45 612 T2, EP 1 272 114 B1, EP 1 570 796 B1, EP 1 088 522 B1, EP 1 143 867 B1, and EP 1 211 994 B1 further constitute the state of the art. In the following, this state of the art is assumed to be known.

In particular, such osteosynthesis devices serve to stabilise the position of bone fragments in the case of bone fractures in order to support the osteosynthesis in the correct relative position of the fragments.

The state of the art according to DE 196 29 011 C2 already provides self-cutting screws which are screwed into an insert around the through holes, which is softer relative to the plate, in particular also in an inclined relation to the longitudinal axis of the through hole.

WO 2007/009124 A2 describes a osteosynthesis plate with movable inserts. The provision of such movable inserts is an approach which is in basic opposition to the present invention because the movable inserts result in a non-fixed arrangement of the insert in the plate.

WO 2006/103245 A1 discloses a ring that is screwed into a plate by means of an outer thread. Such a ring is movable in the plate because it can be screwed more or less into the plate. Therefore, this prior art does also not teach a fixed positioning of the insert in the plate.

EP 1 336 383 A1 describes an insert which is called an “annular ring” or as a “collar”. This ring or collar has a shape that is dependent from the temperature. The collar or ring of this prior art does not solve for screwing a screw. In particular, there is no self-cutting screw provided. Rather, the screw has a smooth part which is arranged in said collar.

WO 00/69351 A teaches (claim 55, FIG. 6) a clamp with a soft inlay.

### SUMMARY OF THE INVENTION

The invention is based on the object to provide an osteosynthesis device of the initially mentioned type, which ensures a high functional reliability when in use while offering simple and economical manufacture.

This is achieved by a positive locking between said insert and the plate in such a manner that the insert is secured (held) in its position in the plate at least against tensile forces between the plate and the bone.

According to the invention, the insert is locked in the plate such that movement in all directions is prevented, in particular, rotational movement and translational movement.

An osteosynthesis plate of the type discussed herein has two sides: One side faces the bone and the opposite side of the plate thus faces away from the bone. The screw is screwed into the through hole from the latter side of the plate, which may also referred to as exterior side, and then protrudes beyond the interior side of the plate, whereby it is screwed into the bone fragment. When the plate is drawn towards the bone in the course of the screwing operation, a “tensile force” in the above sense prevails, and the invention ensures by the mentioned positive locking that the insert whose material allows the screw to create its thread in the insert by self-cutting is not drawn out of the plate towards the bone.

A further embodiment of the invention provides for the positive locking to also position the insert in a stabilised manner in the plate, that is to say that the insert is secured against compressive forces due to the positive locking, i. e. the forces that might urge the insert away from the bone and out of the plate.

In other words, the positive locking between the insert and the plate acts in both directions parallel and anti-parallel to the longitudinal axis of the through hole, i. e. virtually in all directions.

According to a preferred embodiment of the invention, the positive locking has one or several protrusions at the circumference of the insert which engage in complementary recesses in the plate. Either alternatively or in combination with the above mentioned embodiment, it is also possible to provide the plate with protrusions which engage in complementary recesses in the insert.

According to the invention, the insert with its entire surface area is in intimate contact with a complementary surface of the plate. Therefore, no cavities exist between the insert and the plate.

Another embodiment of the invention provides for achieving the positive locking by means of a conical shape of insert and plate. Insert and plate may also be configured in a double-conical shape in such a manner that two conical shapes with opposite directions are provided, which meet approximately in the centre plane of the plate either in such a manner that the tips of the cones are facing one another or are facing away from one another. In the first case, a positive locking in diabolo shape, and in the second case a positive locking in diamond shape will result.

Another embodiment of the invention, which, in particular enables a simple manufacture of the osteosynthesis device, provides for the insert to be a two-piece component.

The plate of the osteosynthesis device consists preferably of a metal, and the insert is made of a material which, although is has a sufficient dimensional stability, enables a screw, when being screwed in, to form its thread in the insert's material by self-cutting. PEEK (polyetheretherketone) was found to be a particularly suitable material for this purpose. Other materials, however, are also suitable, such as synthetic materials with the mentioned properties. Other suitable materials for the insert are, in particular, PE (polyethylene) and PMMA (polymethylmethacrylat), the latter also in the form of bone cement.

The term “plate” in the context of osteosynthesis devices is a technical term and covers, in particular, elongated formations in which through holes are arranged only in a row.

### BRIEF DESCRIPTION OF THE DRAWINGS

In the following, embodiments of the invention will be described with reference to a drawing in which:

FIG. 1 shows a schematic section through a first embodiment of a plate for an osteosynthesis device as a sectional view in the area of a through hole;

FIG. 2 shows a second embodiment of a plate for an osteosynthesis device as a schematic sectional view in the area of a through hole;

FIG. 3 shows a third embodiment of a plate for an osteosynthesis device as a sectional view in the area of a through hole;

FIG. 4 shows a fourth embodiment of a plate for an osteosynthesis device as a schematic sectional view in the area of a through hole;

FIG. 5 shows a fifth embodiment of a plate for an osteosynthesis device as a schematic sectional view in the area of a through hole; and

FIGS. 6 and 7 show a sixth embodiment of a plate for an osteosynthesis device as a schematic sectional view in the area of a through hole with a two-piece insert.

### DETAILED DESCRIPTION OF THE PREFERRED EMBODIMENT

The figures show respective embodiments of plates for an osteosynthesis device, with functionally identical or functionally similar components and features being identified by the same reference numerals, which are supplemented by letters, if required.

The figures do not illustrate the complete plates for the osteosynthesis, but only those areas of the plate which are necessary for the explanation of the invention, i. e. plate areas surrounding a through hole through which a screw (not shown) may be installed. It is understood that each of the mentioned plates generally comprises a plurality of through holes, even if the illustrated embodiments are shown with only one through hole each. The other through holes are designed analogously.

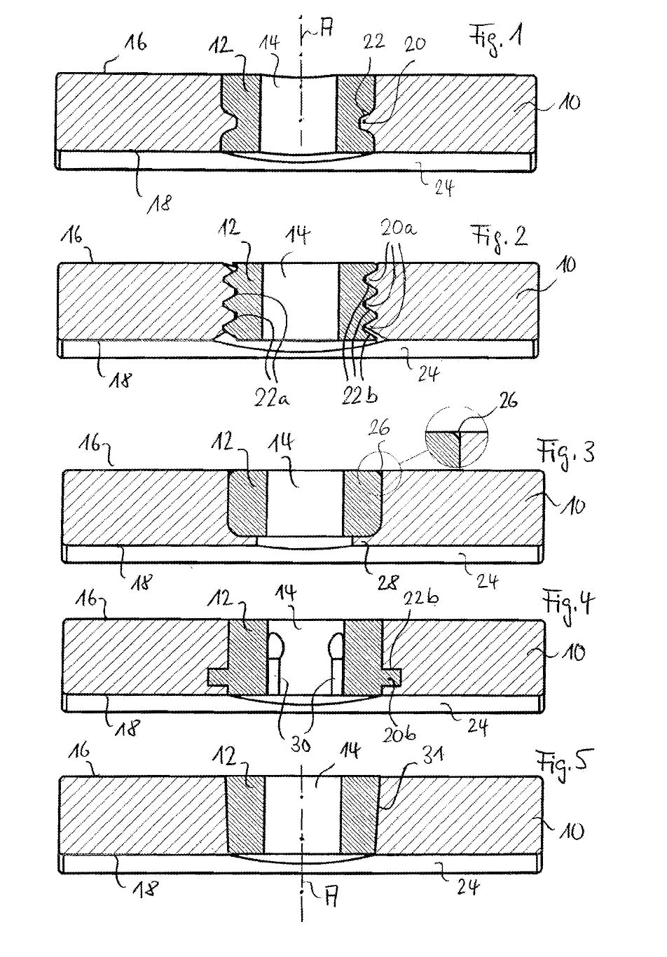

In the first embodiment of an osteosynthesis plate according to FIG. 1, the plate 10 consists of a metal, e. g. titanium. An insert 12 made of PEEK is placed in an opening of the plate 10 and securely positioned therein by positive locking. A through hole 14 is formed in the insert 12, through which a screw (not shown) is screwed, with the screw self-cutting its thread into the material of the insert 12. The longitudinal axis of the circular cylindrical through hole 14 is indicated in FIGS. 1 and 5 as well as in FIG. 6, and extends correspondingly in the examples according to FIGS. 2, 3, and 4. When using the plate 10, the screw may be installed in an inclined position, i. e. at an angle relative to the longitudinal axis A, depending on the surgical situation and the location of the bone fragments.

The plate 10 has an exterior side 16 and an interior side 18. This means that with the intended use of the plate for an osteosynthesis, the interior side 18 faces the bone to be healed. In the figures, the screw is installed from above through the through hole 14 so that the screw head in the installed position of the plate rests on the top of the plate, i. e. on the exterior side 16.

In the embodiment according to FIG. 1, the positive locking of the insert 12 in the plate 10 is achieved by the cooperation of a protrusion 20 at the plate and a recess 22 in the insert 12. In a first variant of this embodiment, the protrusion 20 and the recess 22 may be completely rotation-symmetrical, i. e. when rotating the section shown in the figure about the axis A, each angle of rotation will result in the same image. According to a modified variant, the anti-rotation protection of the insert 12 with respect to the plate 10 may also be provided in a non-complete rotation-symmetrical configuration of the insert 12 and the plate 10. With this variant, the shape of the outer circumference of the insert 12 (and accordingly the complementary shape of the plate 10) in a section perpendicular to the longitudinal axis A of the through hole 14 may not be circular so that a rotation of the insert 12 relative to the plate 10 is prevented. An anti-rotation protection in the above sense may also be achieved by a protrusion (not shown) which is provided at a certain place of the insert 12, which does not extend over the entire circumference but engages in a complementary recess (not shown) in the plate 10 at this place only. As an anti-rotation protection in this sense, several protrusions which are distributed over the circumference may also be contemplated. The two above mentioned variants for providing an anti-rotation protection between the insert 12 and the plate 10 may analogously be applied to all other embodiments of the invention according to FIGS. 2, 3, 4, 5, and 6 which will be described in more detail below.

The embodiment according to FIG. 2 differs from the above described embodiment according to FIG. 1 essentially in that several protrusions 20a are formed on the plate 10 which come into accurate engagement with several complementary recesses 22a in the insert 12 in lieu of the single protrusion 20 and the single recess 22 according to FIG. 1. Otherwise, the embodiment according to FIG. 2 may be derived from the above description of FIG. 1.

In the embodiment according to FIG. 3, the insert 12 is held against tensile forces in the above sense in a positive locking at the interior side 18 of the plate 10 by means of a protruding edge 28 at the interior side 18, while the usually less loaded positive support in the opposite direction at the exterior side 16 of the plate 10 is achieved by a flanged edge 26 of the plate 10.

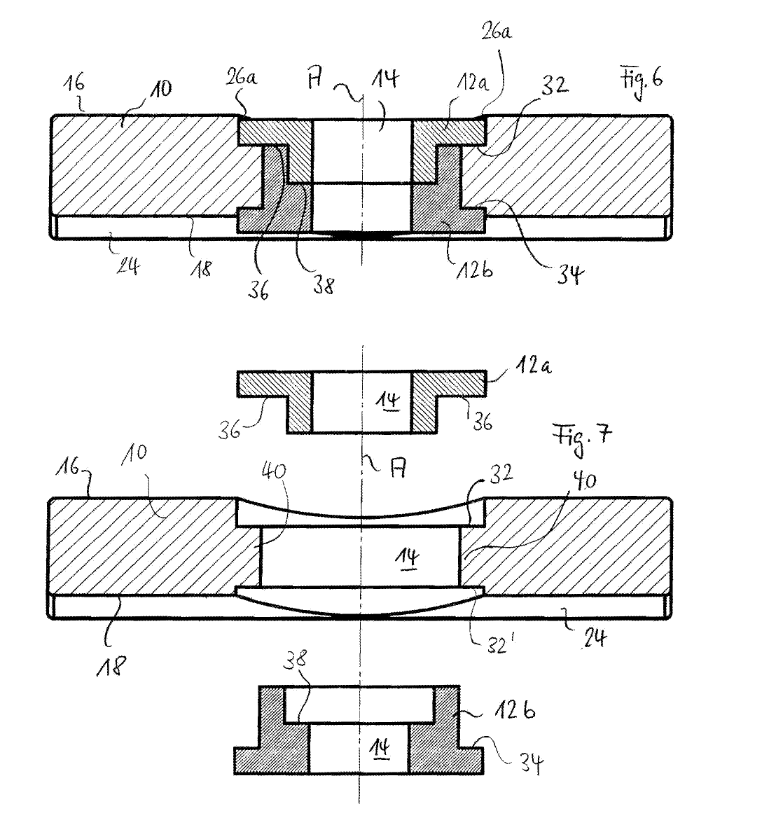

The embodiment according to FIG. 4 shows a modification of the embodiment according to FIG. 1 in that a protrusion 20b is formed at the insert 12 which positively and precisely engages in a recess 22b in the plate 10. In the embodiment according to FIG. 4 there are also provided several slits 30 in the insert 12, which are distributed over the circumference of the insert 12 and which allow the insert to be compressed upon the installation of the insert 12 into the plate 10 in order to facilitate the insertion of the protrusion 20b into the recess 22b.

The embodiment according to FIG. 4 is particularly advantageous in that an insert in the plate may be exchanged, for example, in case of a defective insert, on site and by the user.

In the embodiment according to FIG. 5, the locking of the insert 12 by the positive locking in the plate 12 is achieved by means of the outer shape of the insert 12 whose surface area is formed as a cone 31 which precisely fits a corresponding complementary cone in the plate 10. This enables a positive locking of the insert 12 in the plate 10 against the above defined tensile forces. If the insert 12 is also to be secured by a positive locking in the opposite direction (i. e. upwards in FIG. 5) in the plate 10, then a double cone may be used in the above described sense, i. e. the outer contour of the surface area of the insert 12 may, for example, be diabolo-shaped or, vice versa, the inner contour of the surface of the plate which is in contact with the insert 12 may correspondingly be diamond-shaped.

FIGS. 6 and 7 show another embodiment of an osteosynthesis plate with an insert which here consists of two elements 12a, 12b. FIG. 6 shows the plate with the insert 12 installed, while FIG. 7 shows the same plate in an exploded view (prior to assembly).

As can be clearly seen in detail in FIGS. 6 and 7, steps 32, 34, 36, 38 in the components 12a, 12b of the insert and complementary steps in the plate 10 provide for the positive secure positioning of the two insert components in the plate 10. For the positive locking, a protrusion 40 is formed at the plate 10, which cooperates via steps 32, 32′ with the corresponding complementary steps 34, 36 in the insert components 12a and 12b, respectively. In this embodiment, too, like in all other illustrated embodiments, the positive locking over the surface area of the insert is free from gaps, i. e. no cavities between the insert 12a, 12b and the plate 10 will occur.

In all embodiments, the connection between the insert 12 or 12a, 12, respectively, and the plate 10 may optionally be enhanced by an adhesive.

The positive locking in the above described embodiments between the insert and the plate may be enhanced by non-positive locking, in particular, frictional locking.

In addition, all embodiments may comprise an anti-rotation protection of the type as described above in more detail with reference to FIG. 1.

What is claimed is:

1. An osteosynthesis device, comprising a plate (10) with at least one through hole (14) through which a screw may be screwed into a bone for bridging bone fractures and for joining the bone with the plate (10), wherein the plate comprises an insert (12) around the through hole (14), which insert comprises a material which is softer than that of the plate (10), into which insert the screw may be screwed while at least partially self-cutting a thread, wherein there is a positive locking (20, 22; 30; 32, 34, 36, 38) between the insert (12; 12a, 12b) and the plate (10) in such a manner that the insert is abutting over its entire shell against a complementary surface of the plate and, thereby, the insert is fixed in the plate against movement in all directions.

2. The osteosynthesis device according to claim 1, characterised in that the positive locking (20, 22; 30; 32, 34, 36, 38) between the insert (12; 12a, 12b) and the plate (10) also secures the insert against compressive forces between the plate (10) and the bone.

3. The osteosynthesis device according to claim 1, characterised in that the positive locking (20, 22; 30; 32, 34, 36, 38) comprises one or several protrusions (12′) at the circumference of the insert (12) which engage in one or several, respectively, complementary recesses (22b) in the plate (10).

4. The osteosynthesis device according to claim 1, characterised in that the positive locking (20, 22; 30; 32, 34, 36, 38) comprises one or several protrusions (20a; 28; 40) at the plate (10), which engage in one or several, respectively, recesses (22b) in the insert.

5. The osteosynthesis device according to claim 1, characterised in that the positive locking comprises one or two cones (30).

6. The osteosynthesis device according to claim 1, characterised in that the insert (12a, 12b) is a two-piece component.

7. The osteosynthesis device according to claim 1, characterised in that the insert consists of a synthetic material, in particular, of PEEK (polyetheretherketone).

8. The osteosynthesis device according to claim 1, characterised in that the plate consists of a metal, in particular, of titanium.

9. The osteosynthesis device according to claim 1, characterised in that the insert comprises an anti-rotation protection in such a manner that the insert (12) cannot be rotated about its centre axis (A), and in particular, in such a manner that the insert is not circular in a section perpendicular to its longitudinal axis (A).

10. The osteosynthesis device according to claim 2, characterised in that the positive locking (20, 22; 30; 32, 34, 36, 38) comprises one or several protrusions (12′) at the circumference of the insert (12) which engage in one or several, respectively, complementary recesses (22b) in the plate (10).
